(19) United States
(12) Patent Application Publication
(10) Pub. No.: US 2022/0359037 A1
(43) Pub. Date: Nov. 10, 2022

(54) Non-Invasive Classification of Benign and Malignant Melanocytic Lesions Using MicroRNA Profiling

(71) Applicant: THE REGENTS OF THE UNIVERSITY OF CALIFORNIA, Oakland, CA (US)

(72) Inventors: Robert Judson-Torres, San Francisco, CA (US); Maria Wei, San Francisco, CA (US)

(73) Assignee: THE REGENTS OF THE UNIVERSITY OF CALIFORNIA, Oakland, CA (US)

(21) Appl. No.: 16/980,812

(22) PCT Filed: Mar. 25, 2019
(86) PCT No.: PCT/US2019/023834

§ 371 Date: Sep. 14, 2020

Related U.S. Application Data
(60) Provisional application No. 62/647,616, filed on Mar. 23, 2018.

(51) Int. Cl. G16B 25/10; C12Q 1/6886; G16B 40/20

(52) U.S. Cl. CPC G16B 25/10; C12Q 1/6886; G16B 40/20; C12Q 2600/158; C12Q 2600/178

(57) ABSTRACT

Differentiating benign cutaneous lesions from melanoma is an imprecise and subjective endeavor. The use of micro-RNAs has been investigated, but results have not been consistent across studies and clinical applications are lacking. The invention provides new micro-RNA signatures for differentiating benign lesions from melanoma. The micro-RNA signatures are robust, being stable across detection platforms, diverse sample types, and patient populations. The diagnostic methods based on these signatures control for variations in lesion composition and sample diversity, and permit cross-platform comparisons. The micro-RNA signatures and methods are amenable to the use of samples from convenient non-invasive tape strip biopsy.

### CROSS-REFERENCE TO RELATED APPLICATIONS

This application is a 35 USC § 371 National Stage application of PCT International Application Number PCT/US2019/023834, entitled “Non-invasive classification of benign and malignant melanocytic lesions using microRNA profiling,” filed Mar. 25, 2019, which claims the benefit of priority to U.S. Provisional Application Ser. No. 62/647,616, entitled “Non-invasive classification of benign and malignant melanocytic lesions using microRNA profiling,” filed Mar. 23, 2018; the contents which are hereby incorporated by reference.

### STATEMENT REGARDING FEDERALLY SPONSORED RESEARCH OR DEVELOPMENT

This invention was made with government support under grant number OD019787 awarded by the National Institutes of Health. The government has certain rights in the invention.

### BACKGROUND OF THE INVENTION

The advanced stages of melanoma are associated with five-year survival rates of less than 20% and melanoma is responsible for thousands of deaths each year. Although the disease is curable when detected and treated early, the process of differentiating between malignant lesions and the more prevalent benign lesions is challenging. The definitive diagnosis of concerning lesions is achieved through histopathologic assessment of a biopsy specimen, but a considerable rate of discordance even among expert pathologists has been established. In a large-scale study, interobserver discordance rates as high as 57-75% and intraobserver discordance rates at 37-65% were reported (Elmore et al. 2017. Pathologists' diagnosis of invasive melanoma and melanocytic proliferations: observer accuracy and reproducibility study. BMJ 357: j2813). These observations highlight the complexity and subjectivity of histopathologic assessment and emphasize the need for objective methods for distinguishing malignant from benign lesions to augment current practices.

Molecular biomarkers have the potential to provide robust, objective and quantitative measurements of disease state. One class of candidate biomarkers is small non-coding microRNAs (miRNAs). miRNAs stabilize transcriptional programs and their expression can distinguish cell state transitions during mammalian development and in disease progression. Having a smaller size, reduced complexity, and superior stability over mRNA transcripts, miRNAs are appreciated as potentially valuable candidate biomarkers for many conditions and diseases. However, despite abundant studies focused on a breadth of diseases, few miRNA biomarkers have become applied in the clinical setting. One reason these promising candidate biomarkers have yet to reach their potential is the frequent lack of reproducibility between differential expression studies. Discrepancies in differential expression signatures across comparable studies have been attributed to sample heterogeneity, platform-specific biases in miRNA detection, and an absence of standardized normalization strategies.

Exemplifying these complications are studies that have explored the use of miRNAs as biomarkers for melanoma (for example, as reviewed in: Jarry et al. 2014. The validity of circulating microRNAs in oncology: five years of challenges and contradictions. Mol Oncol 8: 819-829; Jayawardana et al. 2016. Identification, Review, and Systematic Cross-Validation of microRNA Prognostic Signatures in Metastatic Melanoma. J Invest Dermatol 136: 245-254; Margue et al. 213. New target genes of MITF-induced microRNA-211 contribute to melanoma cell invasion. PLoS One 8: e73473; and Raya et al. 2012. Aberrations in the micro-RNA biogenesis machinery and the emerging roles of micro-RNAs in the pathogenesis of cutaneous malignant melanoma. Pigment Cell Melanoma Res 25: 740-757). Independent studies using various platforms (microarray, RT-qPCR array, RNA-seq) to compare miRNA profiles between benign melanocytic lesions and melanomas have resulted in more than 500 different miRNAs identified as significantly differentially expressed. However, only seven of these miRNAs showed reproducible expression differences in at least half of the cohorts, and none were identified in every study. Several of the most reproducibly identified miRNAs—miR-211-5p, miR-125b-5p, and miR-21-5p—have been validated as differentially expressed in benign and malignant pigmented lesions using in situ hybridization, suggesting some miRNAs could function as biomarkers. However, differential expression of these same miRNAs was not observed in 10-30% of cohorts.

Accordingly, there remains a need in the art for the identification of robust miRNA biomarkers to accurately differentiate between precancerous and cancerous lesions, across patient populations, heterogeneous samples, and miRNA detection platforms.

### SUMMARY OF THE INVENTION

The various inventions disclosed herein are based on the identification of novel miRNA signatures that are indicative of benign nevus and melanoma cell types. These miRNA profiles improve upon the prior art in being stable across diverse patient pools and across various detection platforms and provide sensitive indicators of disease state.

The use of miRNAs to distinguish non-cancerous nevi from melanomas has been investigated by many research groups. However, the miRNA signatures derived by these prior art efforts have been of limited clinical use. The novel methods and tools of the invention were developed using new investigative approaches to this problem. First, microdissection of adjacent precursor nevus and descendent melanoma regions was performed, reducing noise in the dataset by limiting the analyses to genotype- and lesion-matched samples and controlling for sample purity. Second, a series of machine learning based analyses were applied to eliminate miRNAs that were influenced by other confounding variables, resulting in optimized models for melanoma diagnosis.

Third, the confounding effects of non-tumor cell contamination in samples comprising melanoma was addressed by the use of a ratio-based based classifier. Contamination by non-tumor cells is expected to vary among samples dependent on their size, histologic type (predominantly junctional versus intradermal), and preparation (e.g. precision of microdissection). If not controlled for, variation in tumor cellularity seems to degrade the reproducibility of miRNA signatures across studies. The ratio-based approach described herein controls for variations in lesion composition (e.g. the relative amount of malignant and benign tissue) when micro-dissection boundaries are not known and differences in tumor cellularity when genetic data are not available. This approach also normalizes for the fraction of miRNAs of melanocytic origin (as opposed to the totality of miRNAs), amplifies the signal from malignant cells by normalizing melanoma-enriched miRNAs to melanoma-depleted miRNAs, and permits cross-platform comparisons without the need for cross-platform normalization.

In a first aspect, the scope of the invention encompasses the use of novel miRNA signatures disclosed herein to differentiate non-cancerous nevi from melanoma.

In a second aspect, the methods of the invention may be applied by the use of non-invasive tape strip assays to collect samples for analysis by the novel miRNA signatures of the invention.

In another aspect, the scope of the invention encompasses tangible products for implementing the novel methods of the invention.

### BRIEF DESCRIPTION OF THE DRAWINGS

FIGS. 1A, 1B, 1C, 1D, and 1E. FIG. 1A is a flow chart summarizing the work-flow for miRNA-Seq classifier development and testing. FIG. 1B is a box plot depicting Boruta feature selection for the top miRNAs for classifying benign nevi from melanoma. FIG. 1C is a box plot depicting normalized miRNA-Seq counts from micro-dissected FFPE samples. Counts from melanoma regions and nevus regions. Boxes indicate mean, first and third quartiles. FIG. 1D depicts ROC curves for cross-platform testing of MiRTM using the combined set of publicly available datasets. Threshold determined from the discovery sequencing set and optimal threshold are shown with corresponding sensitivity and specificity annotated for each. FIG. 1E depicts the Precision-Recall curve for testing of the combined set using MiRTM.

FIGS. 2A, 2B, and 2C. FIG. 2A is a flow chart depicting the workflow for for assembling and testing a randomly selected cohort of melanomas and nevi for the validation cohort. FIG. 2B is an ROC curve for validation cohort using MiRTM. Threshold determined from the discovery sequencing set and optimal threshold are shown with corresponding sensitivity and specificity annotated for each. FIG. 2C is a Precision-Recall curve for testing of the validation cohort using MiRTM.

FIGS. 3A, 3B, and 3C. FIG. 3A and 3C depict the process of tape strip sampling. FIG. 3A depicts a circular tape strip 101 with non adhesive arc 102 for gripping with fingertips, forceps, or the like. FIG. 2B shows the circular tape patch affixed over a nevus 105 on a subject's hand 104. FIG. 3B shows the circular tape patch 101 after removal from the subject with cellular material captured by the adhesive section.

FIG. 4A and FIG. 4B. FIG. 4A depicts the design of diagnostic process of non-invasive miRNA profiling by tape strip sampling. Collection and analysis of tape strip samples from melanocytic lesions are conducted in parallel with standard dermatological care. FIG. 4B depicts a bar plot of the ratio between melanoma-associated miRNAs and nevus-associated mRNAs in a FFPE cohort.

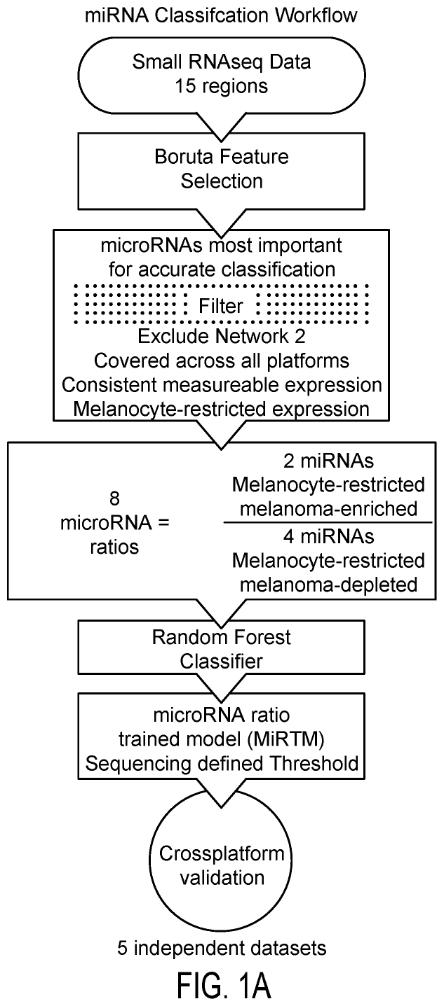

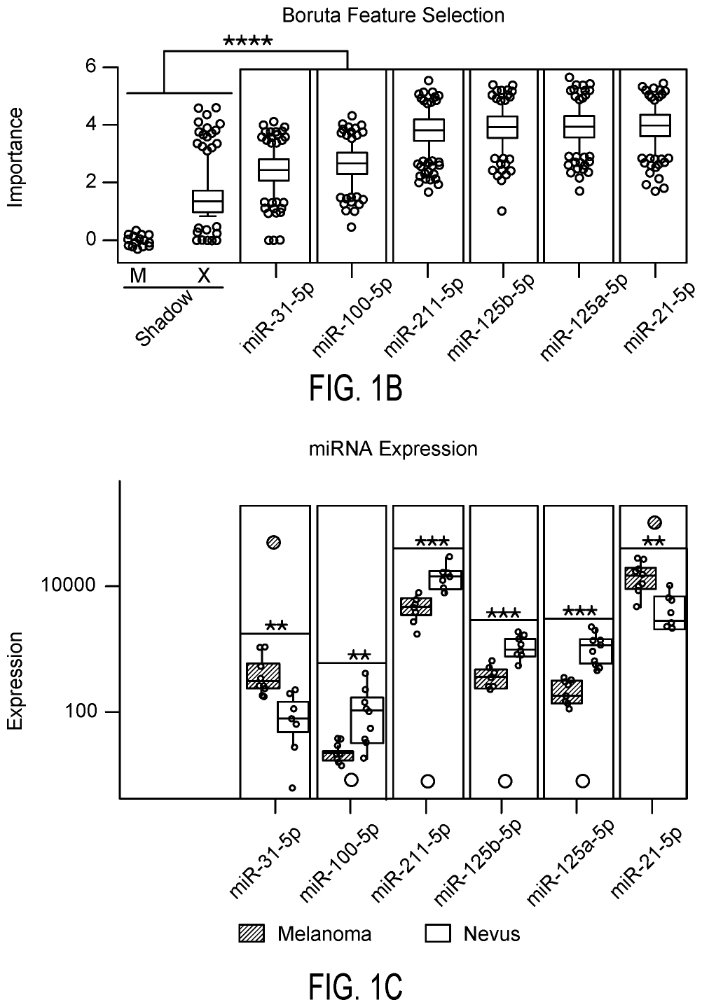

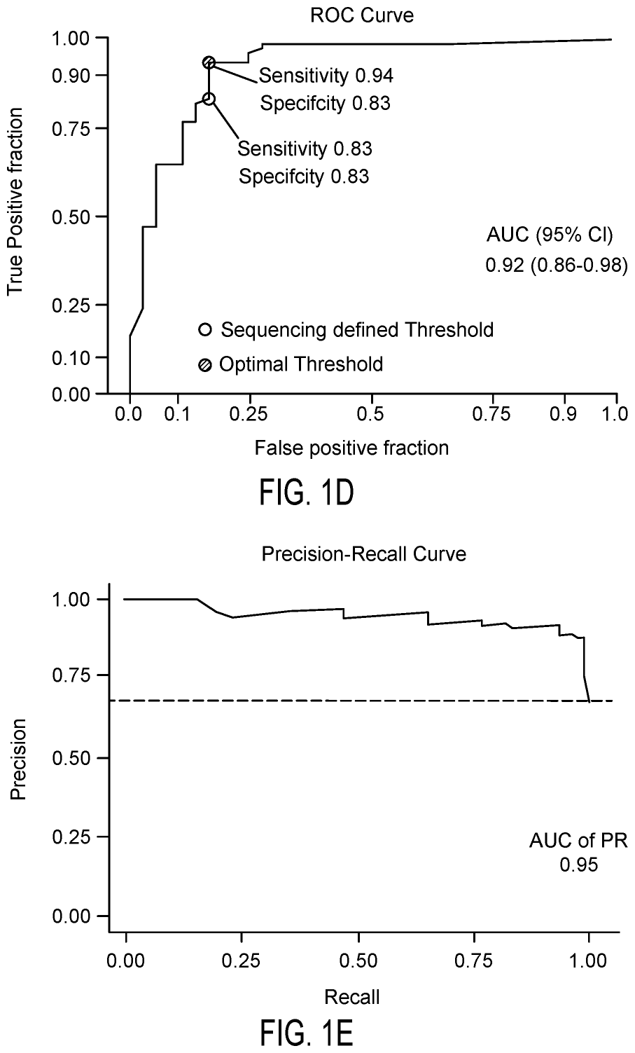

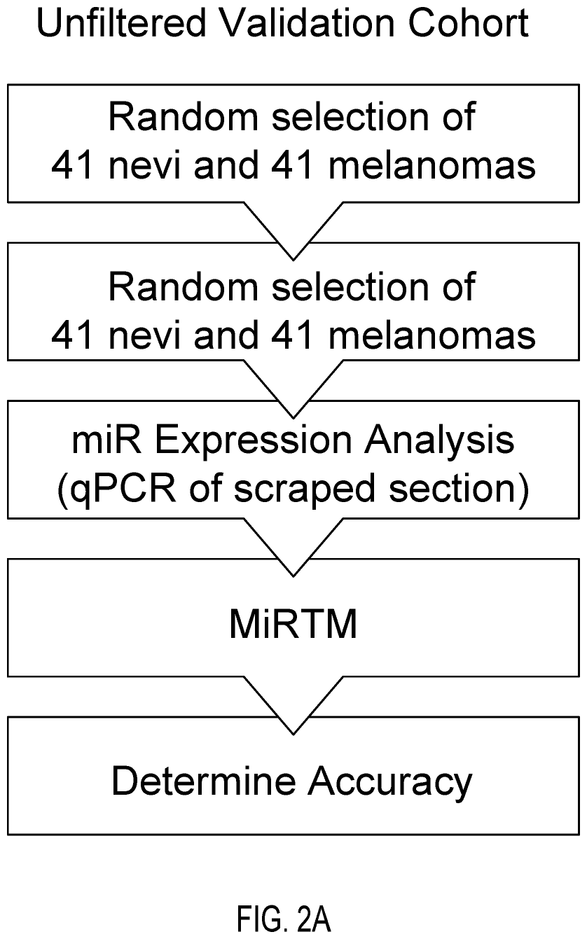

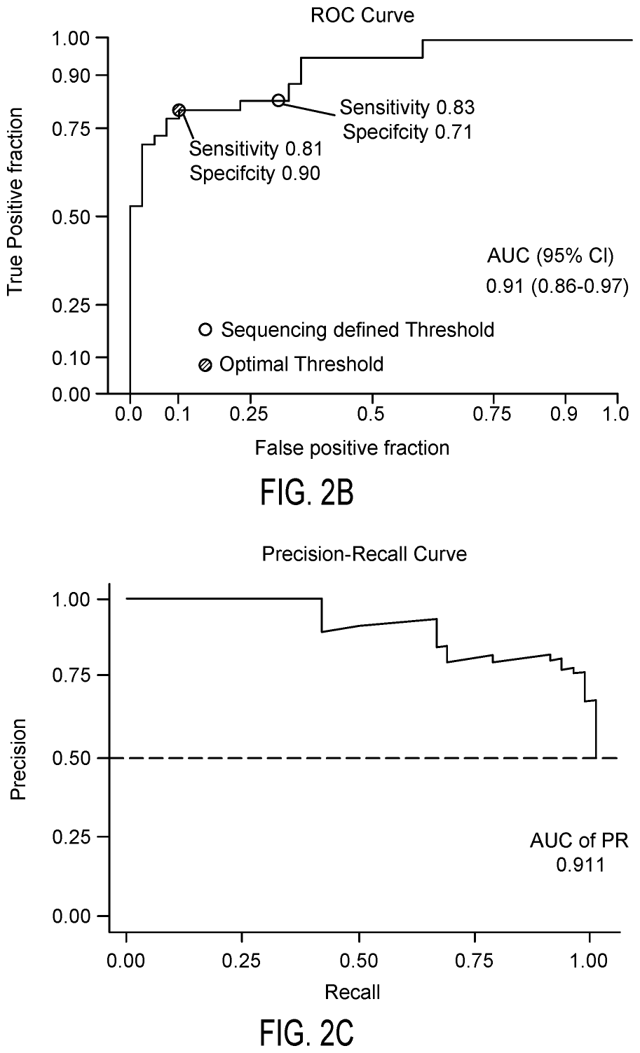

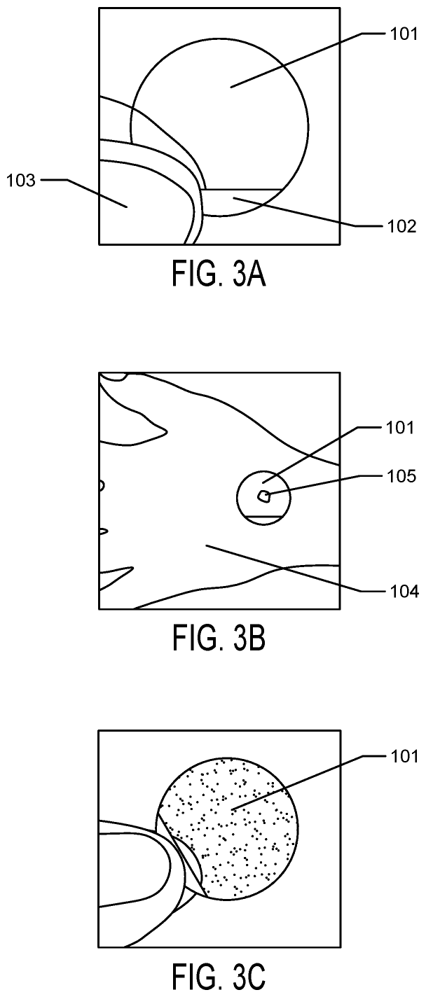

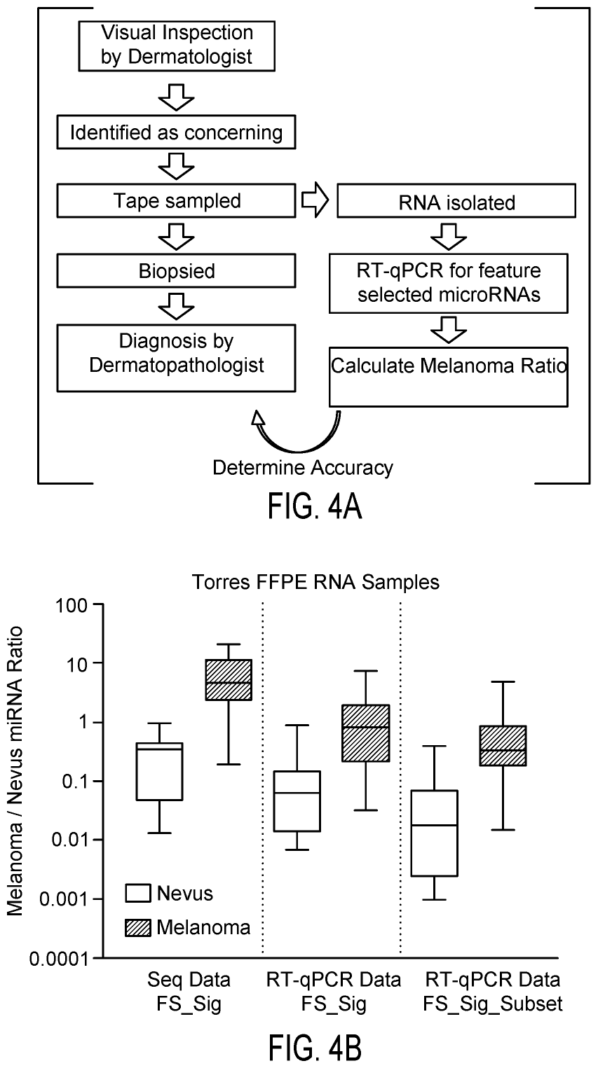

### DETAILED DESCRIPTION OF THE INVENTION

Numerous studies have analyzed miRNA expression at different stages of melanoma progression, collectively identifying over 500 miRNAs enriched in nevi or melanomas. Herein, the expansive list of putative benign/melanoma miRNA biomarkers known in the art has been reduced to a small number of miRNAs that reproducibly distinguish nevi from melanoma across profiling platforms. The novel miRNA signatures of the invention were developed by controlling two important variables, interobserver variability of diagnosis and variability in tumor cellularity. To address the first variable, a discovery cohort was utilized for which the diagnosis accuracy was established by requiring the median concordance among five to eight different dermatopathologists and supporting genetic features, eliminating the possibility of training the model with misdiagnosed cases. Secondly, melanomas and nevi were matched as pairs representing different progression stages of the same neoplastic clone, eliminating variability by comparing different lesions from different patients. Thirdly, miRNAs were identified and excluded from the signature whose expression is influenced by tumor cellularity, thereby removing a covariate that has confounded previous analyses. When tested on six datasets assembled by independent groups, a model trained on expression ratios of the refined signature classified benign from malignant melanocytic lesions with an average AUC of ROC above 0.91.

The methods of the invention are applied in differentiating non-cancerous lesions (benign nevi) from cancerous lesions comprising melanoma. Benign nevi, as referred to herein, is used as known in the art and encompasses, for example, any number of non-cancerous skin lesions, including common moles, dysplastic nevi, “atypical” nevi, and other lesions. As used herein, “melanoma” is used as known in the art, for example, referring to any malignant or cancerous condition of the melanocytes, including radial or invasive neoplasms.

The general method of the invention comprises a method of classifying a cutaneous lesion sample as comprising either melanoma or non-cancerous tissue, by the following steps:
a sample is collected comprising cutaneous lesion tissue of a subject;
the sample is assayed, wherein the expression values of selected melanoma miRNA biomarkers and of selected benign lesion miRNA biomarkers are assessed;
the measured expression values of the selected melanoma miRNA biomarkers and the selected benign lesion miRNA biomarkers are input to a classifier model; and
a classification of the sample as melanoma or benign is output by the model.
   The various elements of the general method are next described in detail.

### Samples

The selected miRNA biomarkers will be measured in a sample. The sample may comprise any relevant sample for assessment of miRNAs in a melanocytic lesion. In one embodiment, the sample comprises biopsied tissue, for example, formalin-fixed paraffin-embedded (FFPE) lesion samples, as known in the art. Biopsied tissue may include tissue collected by shave biopsy, saucerization excision, punch biopsy, elliptical excision or incisional samples.

### Tape Strip Sample Collection

In one implementation, the sample comprises a tape strip sample, as known in the art. In tape strip sampling, a tape or patch comprising an adhesive is applied to a cutaneous lesion and removed, wherein lesion surface cells and cellular debris/exudates are captured by and embedded within the adhesive. miRNAs captured in the collected material may be liberated from the tape strip by appropriate treatment and subsequently quantified. This method provides an extremely convenient and non-invasive means of collecting miRNAs.

Exemplary tape strip sampling products include the DSQUAME D100™ striping disc (CUDerm, Dallas Tex.). The adhesive of the tape will preferably comprise an adhesive inert to biomolecules including nucleic acids such as DNA and RNA. The adhesives may comprise, for example, acrylic-based, cyanoacrylate-based, urethane-based, polyurethane-based, polypropylene-based, polyester-based, polyisobutylene-based, and silicone-based adhesives.

In certain embodiments, the skin surface to be tested is prepared for sampling by removing hairs on the skin, cleansing the surface with antimicrobial substances, drying the sampling surface, and applying the adhesive tape or patch to the surface. In certain embodiments, the sampling of the skin lesion includes applying and removing one or more times the adhesive tape. In one embodiment, the samples obtained on an adhesive tape may be frozen prior to being analyzed.

Example tape strip sampling products include, for example, adhesive sampling systems described U.S. Pat. No. 5,088,502, incorporated herein by reference in its entirety. Another exemplary tape strip product includes, for example, adhesive skin collection kits described by DermTech (La Jolla, Calif.), as further described for example, in U.S. Pat. Nos. 7,183,057, 7,297,480, or 6,949,338, In yet another example, a tape strip product includes, for example, an adhesive skin collection method as described in U.S. Patent Publication No. 2007/0281314.

### Micro RNA Expression Measurement

The various embodiments of the invention are based on the measurement of selected miRNAs. Micro RNAs, as known in the art, are small, non-coding regulatory RNAs, typically 22 nucleotides in length. Reference to specific miRNAs herein will refer to established sequences as named in the MIRBASE database, as known in the art.

Advantageously, the miRNAs identified for use in the methods of the invention are robust biomarkers that are uniformly and highly expressed in either melanomas or benign lesions, and which are consistently measurable by various miRNA assay techniques. Accordingly, the methods of the invention may be achieved by any number of measurement platforms, with dependable results across detection systems.

In one implementation, miRNA expression levels are measured by microarray analysis, as known in the art. Typical array platforms comprise addressed probes immobilized on a substrate, the probes comprising nucleic acid sequences complementary to their target miRNAs. Arrays are probed by exposing fluorescently labeled RNA derived from samples to the immobilized probes, wherein target sequences will hybridize complementary probes if present. Quantification of the miRNAs is achieved by fluorescence analysis. Any type of miRNA microarray may be utilized in the methods of the invention.

In another implementation, target miRNA expression levels are measured by real time quantitative PCR (RT-qPCR), as known in the art. In one implementation of this technique, target miRNAs in a sample are amplified using sequence-specific primers, with fluorescent or other labels, such as Taq polymerase probes, for example, TAQMAN™ probes (Thermo Fisher, Inc.), intercalating dyes, for example SYBRGREEN™ (Qiagen Corp.), or hairpin molecular beacons, used to quantify the amplification product.

In another implementation, target miRNA expression levels are measured by RNA sequencing (RNA-Seq), also called whole transcriptome shotgun sequencing, as known in the art. In one implementation, miRNAs in the sample are first isolated through size selection, then linkers are added to the 3′ and 5′ ends. cDNAs are then generated by reverse transcription, followed by sequencing with next-generation sequencing tools. Examples of next-generation sequencing platforms include Illumina systems and the TRUSEQ™ Small RNA Library Prep Kit (Illumina, Inc.).

Other methods of miRNA measurement may also be used, for example, multiplex RNA profiling, Northern blot, or other means known in the art.

The product of the miRNA measurement step is an expression value. The expression value of a selected miRNA may be any qualitative or quantitative measure of the miRNA. For example, quantitative measures of target miRNA abundance include for example, absolute values, signal values (e.g. raw fluorescence counts), relative values (e.g. fold change compared to a control or baseline level), and/or normalized values. For example, by arrays, expression may be measured as fluorescence intensity, with RT-qPCR, expression may be measured as a Ct value, and with sequencing, expression may be measured as read counts. In one implementation, read counts are utilized to quantify expression. Quantitative measures may be log transformed. Alternatively, in some implementations, a qualitative assessment of a selected miRNA is made, wherein the presence or absence of the miRNA in the sample is determined.

As above, quantification values may be normalized, for example, normalized against total RNA, normalized against expression values of other specific individual or sets of miRNAs, including miRNAs specifically expressed in melanocytes, normalized against measurements of lesions size, including area of lesion, total tissue, or total cell number or quantile normalization. For the ratio-based classifiers, described below, the ratios of different expression values of are used, which acts as a built-in normalization, e.g., melanoma expression values normalized against a specific set of miRNAs, comprising benign nevus miRNAs. By use of ratios, units are removed and the expression values are normalized. This allows the same model to work across different miRNA detection platforms.

It will be understood that the scope of the invention extends to proxies of the enumerated miRNAs, a proxy for a selected miRNA being any species whose expression or abundance is highly correlated with the expression or abundance of the selected miRNA. For example, miRNA precursors including pri-miRNAs and pre-miRNAs may be used as proxies for mature miRNAs. Likewise, a tightly co-expressed miRNA may be substituted as a proxy for a selected miRNA. Similarly, miRNAs with an identical mature sequence, or a sequence too similar to be distinguished by the detection platform being used, may also serve as proxies.

### Benign Lesion miRNA Biomarkers

In one aspect, the scope of the invention encompasses a number of miRNAs found to be robust biomarkers that are highly and consistently enriched in benign lesions. These benign lesion miRNA biomarkers, as referred to herein, are miRNA biomarkers associated with melanocytic lesions, also known as melanocytic nevi or nevocytic nevi, wherein the tissue has not progressed to a malignant cancerous condition comprising melanoma. The benign lesion miRNAs of the invention are set forth in Table 1. Each miRNA is listed by its identification number. It will be understood that each enumerated miRNA includes both the 5p and 3p sequences. For example, “hsa-mir-211” describes both hsa-mir-211-5p and hsa-mir-211-3p. The -5p and -3p are different sequences and may be measured independently. These are typically very close proxies for each other as they are, by definition, under the same transcriptional regulatory program. Reference to the use of a specific miRNA -5p or -3p sequence made herein will be understood to encompass the alternative use of the complementary form as well.

TABLE 1
Benign Lesion miRNAs

hsa.miR-211
hsa.miR-328
hsa.miR-125a
hsa.miR-125b
hsa.miR-let.7c
hsa.miR-100
hsa.miR-let.7b
hsa.miR-214
hsa.miR-193a
hsa.miR-6087
hsa.miR-320a
hsa.miR-423
hsa.miR-let.7d
hsa.miR-let.7e
hsa.miR-197
hsa.miR-99b
hsa.miR-532
hsa.miR-574

A “suite” of miRNAs, as used herein, refers to a group comprising one or more miRNAs. In various embodiments, the invention encompasses the use of suites comprising one or more benign lesion miRNAs selected from Table 1: hsa.miR-211, hsa.miR-328, hsa.miR-125a, hsa.miR-125b, hsa.miR-1et7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574. In various implementations, the suite comprises any two, three, four, five, six, seven, eight, nine, ten, eleven, twelve, thirteen, fourteen, fifteen, sixteen, or all of the enumerated miRNAs in Table 1.

In one embodiment, the suite of benign lesion miRNAs comprises miR-211, miR-125a, miR-125b, and miR-100, for example, miR-211-5p, miR-125a-5p, miR-125b-5p and miR-100-5p.

### Melanoma miRNA Biomarkers

The scope of the invention further encompasses a number of miRNAs found to be robust biomarkers of melanoma. As used herein, a melanoma miRNA biomarker is an miRNA that is highly expressed and enriched in melanocytic tissues that have progressed to melanoma. The melanoma miRNAs of the invention are set forth in Table 2. Notably, three of the miRNA biomarkers of melanoma, miRNA-93, miRNA-107, and miRNA-128, are completely novel in the context of melanoma detection.

TABLE 2
Melanoma miRNAs

hsa.miR-17
hsa.miR-93
hsa.miR-107
hsa.miR-22
hsa.miR-31
hsa.miR-98
hsa.miR-21
hsa.miR-128
hsa.miR-185
hsa.miR-7
hsa.miR-15a
hsa.miR-16
hsa.miR-27a
hsa.miR-155
hsa.miR-142
hsa.miR-9
hsa.miR-509
hsa.miR-106b
hsa.miR-148b
hsa.miR-103a
hsa.miR-20a
hsa.miR-25

Certain implementations encompass the use of suites of selected melanoma miRNA biomarkers of Table 2: hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, and hsa.miR.25. The suite of melanoma miRNA biomarkers, may comprise, for example, any two, three, four, five, six, seven, eight, nine, ten, eleven, twelve, thirteen, fourteen, fifteen, sixteen, seventeen, eighteen, nineteen, twenty, twenty-one, twenty-two, or all of the enumerated miRNAs.

In one embodiment, the suite of melanoma markers comprises miR-31 and miR-21, for example, miR-31-5p and miR-21-5p.

In certain implementations of the invention, a tape strip biopsy is utilized to attain the sample. Certain miRNAs biomarkers are more prevalent in tape biopsy samples than tissue samples such as FFPE tissue blocks. miRNA biomarkers enriched in benign lesions and detectable in samples obtained by tape strip sampling are listed in Table 3. In one embodiment, the scope of the invention encompasses the use of a suite of benign miRNA biomarkers selected from Table 3: hsa-miR-3141, hsa-miR-6087, hsa-miR-5010 (for example, hsa-miR-5010-5p), hsa-miR-7847 (for example, hsa-miR-7847-3p), hsa-miR-6089, hsa-miR-5100, hsa-miR-1246, hsa-miR-3960, hsa-miR-6515 (for example, hsa-miR-6515-5p), hsa-miR-6734 (for example, hsa-miR-6734-5p), hsa-miR-4466, hsa-miR-193b (for example, hsa-miR-193b-5p), hsa-miR-4516, hsa-miR-5187 (for example, hsa-miR-518′7-5p), hsa-miR-150 (for example, hsa-miR-150-3p), hsa-miR-7704, hsa-miR-4492, hsa-miR-6512 (for example, hsa-miR-6512-5p), hsa-miR-4532, hsa-miR-4497, hsa-miR-296 (for example, hsa-miR-296-3p), hsa-miR-1290, hsa-miR-30c-1 (for example, hsa-miR-30c-1-3p), hsa-miR-342 (for example, hsa-miR-342-5p), hsa-miR-3196, hsa-miR-629 (for example, hsa-miR-629-5p), hsa-miR-4429, hsa-miR-130b (for example, hsa-miR-130b-3p), and hsa-let-7b (for example, hsa-let-7b-5p). The suite may comprise any two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, or all of the enumerated miRNAs.

TABLE 3
Tape Strip Benign miRNAs

hsa-miR-3141
hsa-miR-6087
hsa-miR-5010
hsa-miR-7847
hsa-miR-6089
hsa-miR-5100
hsa-miR-1246
hsa-miR-3960
hsa-miR-6515
hsa-miR-6734
hsa-miR-4466
hsa-miR-193b
hsa-miR-4516
hsa-miR-5187
hsa-miR-150
hsa-miR-7704
hsa-miR-4492
hsa-miR-6512
hsa-miR-4532
hsa-miR-4497
hsa-miR-296
hsa-miR-1290
hsa-miR-30c-1
hsa-miR-342
hsa-miR-3196
hsa-miR-629
hsa-miR-4429
hsa-miR-130b
hsa-let-7b

In one embodiment, the scope of the invention encompasses the use of a suite of benign miRNA biomarkers selected from Table 1 and/or Table 3, for example, selected from the group consisting of assessing the expression values for each miRNA of a suite of benign miRNA biomarkers selected from hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b.

miRNA biomarkers enriched in melanoma and detectable in samples obtained by tape strip sampling are listed in Table 4. In one embodiment, the scope of the invention encompasses the use of a suite of melanoma miRNA biomarkers selected from Table 4: hsa-let-7g (for example, hsa-let-7g-5p), hsa-miR-31 (for example, hsa-miR-31-5p), hsa-miR-181a (for example, hsa-miR-181a-5p), hsa-miR-28 (for example, hsa-miR-28-3p), hsa-miR-25 (for example, hsa-miR-25-3p), hsa-miR-497 (for example, hsa-miR-497-5p), hsa-miR-140 (for example, hsa-miR-140-3p), hsa-miR-29c (for example, hsa-miR-29c-3p), hsa-miR-181b (for example, hsa-miR-181b-5p), hsa-miR-455 (for example, hsa-miR-455-3p), and hsa-miR-30a (for example, hsa-miR-30a-3p). The suite may comprise any two, three, four, five, six, seven, eight, nine, ten, eleven, or all of the enumerated miRNAs.

TABLE 4
Tape Strip Melanoma miRNAs

hsa-let-7g
hsa-miR-31
hsa-miR-181a
hsa-miR-28
hsa-miR-25
hsa-miR-497
hsa-miR-140
hsa-miR-29c
hsa-miR-181b
hsa-miR-455
hsa-miR-30a

In one embodiment, the scope of the invention encompasses the use of a suite of melanoma miRNA biomarkers selected from Table 2 and/or Table 4: hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, hsa.miR.25, hsa-let-7g, hsa-miR-31, hsa-miR-181a, hsa-miR-28, hsa-miR-25, hsa-miR-497, hsa-miR-140, hsa-miR-29c, hsa-miR-181b, hsa-miR-455, and hsa-miR-30a.

### Classifier Model

The selected miRNA biomarkers are measured in the sample to obtain their expression values. These measurements are then input to a classifier model, wherein the output of the model is, for example, a two-way classification of the sample as being benign, or comprising melanoma. The classifier model may comprise any predictive or classification model based upon a suite of benign lesion miRNA biomarkers selected from Table 1 and a suite of melanoma miRNA biomarkers of selected from Table 2. Alternatively, the classifier model may comprise any predictive or classification model based upon a suite of benign lesion miRNA biomarkers selected from Table 3 and a suite of melanoma miRNA biomarkers of selected from Table 4.

In some implementations, the model inputs include additional variables. In one embodiment, the additional variables comprise the expression values of other miRNAs not listed in Table 1, Table 2, Table 3, or Table 4. The other variables may include, for example patient medical and demographic data such as age, family history of melanoma, previous incidence of melanoma, and other health and demographic factors. In certain implementations, the model utilizes the measurement of additional, non-miRNA biomarkers, for example, descriptive data derived from digital images of the lesions.

Certain miRNA biomarkers of the invention are miRNA biomarkers previously implicated in melanoma. In one aspect, the novelty of the invention is based on the identification of a compact subset of known, putative miRNA biomarkers for differentiating benign lesions from melanoma. The use of a smaller set of miRNA biomarkers advantageously simplifies the miRNA analysis and decreases potential sources of variability. Accordingly, in certain implementations of the invention, the classifier model is based on a selected set of miRNA biomarkers from Table 1 and Table 2 and does not take into account any other miRNA biomarkers. The model may take into account other, non-miRNA biomarkers as well as subject demographic and medical factors and measurements of non-miRNA biomarkers. In one embodiment, the selected suite of benign miRNA biomarkers consists of, or consists essentially of, one or more biomarkers selected from Table 1. In one embodiment, the selected suite of melanoma miRNA biomarkers consists of, or consists essentially of, one or more biomarkers selected from Table 2.

Likewise, in certain implementations of the invention, for example tape strip sampling methods, the classifier model is based on a selected set of miRNA biomarkers from Table 1 and/or Table 3 and Table 2 and/or Table 4 and does not take into account any other miRNA biomarkers. The model may take into account other, non-miRNA biomarkers as well as subject demographic and medical factors and measurements of non-miRNA biomarkers. In one embodiment, the selected suite of benign miRNA biomarkers consists of, or consists essentially of, one or more biomarkers selected from Table 1 and/or Table 3. In one embodiment, the selected suite of melanoma miRNA biomarkers consists of, or consists essentially of, one or more biomarkers selected from Table 2 and/or Table 4.

The classifier model may comprise any type of model, including models derived from machine learning methods, artificial neural networks, support vector machines, weighted voting on data patterns, classification and regression trees, k-nearest neighbor classification, and logistic regression. Classifier models using the enumerated miRNA biomarkers may be generated by one of skill in the art from a plurality of samples comprising known benign nevus melanocytes and known melanoma cells. Alternatively, the miRNA expression data used to generate the classifier may comprise publicly available miRNA expression data, for example, as described in the Examples herein, or in Xu et al. 2012. Differential expression of microRNAs during melanoma progression: miR-200c, miR-205 and miR-211 are downregulated in melanoma and act as tumour suppressors. Br J Cancer 106: 553-561; Jukic et al. 2010. Microrna profiling analysis of differences between the melanoma of young adults and older adults. J Transl Med 8: 27; Sand et al. 2013. Comparative microarray analysis of microRNA expression profiles in primary cutaneous malignant melanoma, cutaneous malignant melanoma metastases, and benign melanocytic nevi. Cell Tissue Res 351: 85-98; Komina et al. 2016 Antiproliferative and Pro-Apoptotic Effects of MiR-4286 Inhibition in Melanoma Cells. PLoS One 11: e0168229; Kozubek et al. 2013. In-Depth Characterization of microRNA Transcriptome in Melanoma. PLoS One 8: e72699; Hanniford et al. 2015; Latchana et al. 2017. Identification of metastasis-suppressive microRNAs in primary melanoma. J Natl Cancer Inst 107; and Chen et al. 2011. miR-193b Regulates Mcl-1 in Melanoma. Am J Pathol 179: 2162-2168.

Machine learning and other classifier model generation techniques for diagnostic biomarker analysis are readily available to the skilled artisan, for example, as described herein or in: U.S. Pat. No. 9,773,308, entitled “Systems and methods for generating biomarkers based on multivariate classification of functional imaging and associated data,” by Silbersweig et al.; U.S. Patent Application Publication Number 20090006055, entitled “Automated Reduction of Biomarkers,” by Fung et al.; U.S. Patent Application Publication Number 20150220838, entitled, “Systems and methods relating to network-based biomarker signatures,” by Martin et al.; and U.S. Patent Application Publication Number 20160032395, entitled “Cancer biomarkers and classifiers and uses thereof,” by Davicioni et al.

The classifier model may comprise an algorithm, wherein biomarker expression data is input and the output comprises a score, the value of which results in classification as benign or melanoma. The inputs may further include selected thresholds, including probability thresholds, sensitivity thresholds, specificity thresholds, or statistical significance thresholds, wherein the output classification will fall within the specified thresholds. Likewise, the classification output may comprise a probability score (e.g. Z-score) or other score indicating the statistical significance or likelihood of the classification being correct. A cut off value that provides the desired sensitivity and specificity of the application may be chosen based upon the training sets.

In one implementation, the inventors of the present disclosure have advantageously developed a simplified transcript ratio classifier model. In this implementation, the classification is derived from a ratio calculated as: the mean expression value of a suite of melanoma miRNA biomarkers selected from Table 2 (or Table 4 in certain implementations) divided by the mean expression value of a suite of benign lesion miRNA biomarkers selected from Table 1 (or Table 3 in certain implementations). The calculated ratio may then be compared against a cutoff value, wherein the sample is classified as a melanoma sample if the ratio exceeds the cutoff value. The cutoff value may be selected based upon desired stringency of the classification, and/or may be adjusted based on different cutoff values optimized for different miRNA detection platforms (e.g. array vs. qPCR techniques).

In one embodiment, in the ratio calculations, the average benign nevus miRNA value may comprise the sum of all miRNA expression values of the miRNAs of the selected benign nevus miRNA suite, divided by the number of miRNAs in the suite. Likewise, the average melanoma miRNA value may comprise the sum of all miRNA expression values of the miRNAs of the selected melanoma suite, divided by the number of miRNA's in the suite. In one embodiment, the ratio is calculated as

$$\frac{[ \frac{{\sum }_{i = 1}^{n} M i}{n} ]}{[ \frac{{\sum }_{j = 1}^{x} B j}{x} ]}$$

wherein n is the number of melanoma siRNAs in the selected suite and M₁ to Mₙ are the values of each melanoma miRNAs in the suite, and wherein x is the number of benign miRNA in the selected suite and B₁ to Bₓ are the expression values of each benign miRNA in the selected suite.

Raw signal values may be transformed. For example, raw signal values expressed as number of counts may be transformed by a factor of 2^{(#counts)}. Expression values may be log transformed. In one implementation, the miRNA expression values are unweighted. In alternative implementations, the miRNA expression values may be weighted.

The resulting ratio may then be compared against a cutoff value. For example, in one embodiment, the cutoff value is determined as the optimal value for accurately differentiating benign from malignant samples in a training set.

### Exemplary Embodiment

In one embodiment, the selected suite of melanoma miRNAs comprises miR-31-5p and miR-21-5p, and the selected suite of benign miRNAs comprises miR-211- 5p, miR-125a-5p, miR-125b-5p, and miR-100-5p. In one implementation, the classifier is based on and uses as inputs the ratio of each melanoma miRNA biomarker expression value divided by each benign miRNA biomarker expression value, for a total of eight ratios: (miR-31-5p/miR-211-5p), (miR-31-5p/miR-125a-5p), (miR-31-5p/miR-125b-5p), (miR-31-5p/miR-100-5p), (miR-21-5p/miR-211-5p), (miR-21-5p/miR-125a-5p), (miR-21-5p/miR-125b-5p), and (miR-21-5p/miR-100-5p). These eight ratios may be used to train a classifier, for example, a random forest classifier.

### Diagnosis

The classification methods of the invention may be augmented with other methods of diagnosing melanoma, for example, inspection of samples by a dermatopathologist, to arrive at a final diagnosis.

The implementations of the invention described herein are directed to the diagnosis of melanoma in human subjects. However, it will be understood that the methods of the invention may be implemented to diagnose melanoma or equivalent conditions in non-human species, including test animals and veterinary subjects, using homologous miRNAs of the selected non-human species.

### Methods of Treatments

In one implementation, the scope of the invention encompasses a method of treating melanoma in a subject, the method of treatment comprising:
a sample is collected from the subject comprising cutaneous lesion tissue;
the sample is assayed, wherein the expression value of selected melanoma miRNA biomarkers and of selected benign lesion miRNA biomarkers is assessed;
the measured expression values of the selected melanoma miRNA biomarkers and the selected benign lesion miRNA biomarkers are input to a classifier model;
a classification of the sample as melanoma or benign is output by the model; and
an appropriate treatment is administered to the subject if the sample is classified as melanoma.

The appropriate treatment may comprise any treatment suitable for the diagnosis, staging, or treatment of melanoma. For example, appropriate treatments may include: staging of the melanoma, assessment of metastasis, surgical removal of the lesion and surrounding tissue, for example, wide local excision, adjuvant treatment, chemotherapy, radiation, and/or immunotherapy.

### Diagnostic Tools

The scope of the invention further extends to compositions of matter comprising diagnostic tools and kits specially configured for performing the methods disclosed herein. The compositions of matter may include agents and compositions for the measurement of of miRNA biomarkers, for example, for the measurement of suites of miRNAs selected from Table 1 and Table 2 or from Table 3 and Table 4. For example, in one embodiment, the agent comprises a microarray directed to the quantification of the target miRNAs. In another embodiment, the agent comprises primers sets for the amplification of the target miRNAs. In another embodiment, the agents comprise components of an RNA-seq assay for detection of the target miRNAs, for example a library preparation kit optimized for the amplification and/or detection of the miRNAs selected from Table 1 and Table 2, or Table 3 and Table 4, comprising, for example, tags for tagmentation, sequencing chips, sequences for primer binding, indices, terminal sequences and primer coated beads.

In one embodiment, the invention comprises a microarray, comprising probes that selectively hybridize with one or more miRNAs from:
a suite of benign miRNA biomarkers selected from the group consisting of hsa.miR-211, hsa.miR-328, hsa.miR-125a, hsa.miR-125b, hsa.miR-1et7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574; and
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, and hsa.miR.25.

In one embodiment, the microarray does not contain probes for other miRNAs, i.e. miRNAs not enumerated in Tables 1 and 2.

In one embodiment, the microarray comprises probes that selectively hybridize with miRNa-21, miRNA-31, miRNA-211, miRNA-100, miRNA-125a, and miRNA-125b. In one embodiment, the microarray only detects miRNa-21, miRNA-31, miRNA-211, miRNA-100, miRNA-125a, and miRNA-125b.

In one embodiment, the microarray comprises probes that selectively hybridize with one or more miRNAs from
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, hsa.miR.25, hsa-let-7g, hsa-miR-31, hsa-miR-181a, hsa-miR-28, hsa-miR-25, hsa-miR-497, hsa-miR-140, hsa-miR-29c, hsa-miR-181b, hsa-miR-455, hsa-miR-30a; and
a suite of benign miRNA biomarkers selected from hsa.miR-211, hsa.miR-328, hsa.miR-125a, hsa.miR-125b, hsa.miR-1et7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b.

In one embodiment, the microarray does not contain probes that hybridize with other miRNAs, i.e. miRNAs not enumerated in Table 1, Table 2, Table 3, or Table 4.

In one embodiment, the invention is a kit, comprising PCR primers for the selective amplification of one or more miRNAs from
a suite of benign miRNA biomarkers selected from the group consisting of hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574; and
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, and hsa.miR.25.

In one embodiment, the kit does not contain primers for the amplification of other miRNAs, i.e. miRNAs not enumerated in Table 1 or Table 2.

In one embodiment, the kit comprises PCR primers for the selective amplification of miRNa-21, miRNA-31, miRNA-211, miRNA-100, miRNA-125a, and miRNA-125b. In one embodiment, the kit comprises PCR primers only for the selective amplification of miRNa-21, miRNA-31, miRNA-211, miRNA-100, miRNA-125a, and miRNA-125b.

In one embodiment, the kit comprises PCR primers for the selective amplification of one or more miRNAs from
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, hsa.miR.25, hsa-let-7g, hsa-miR-31, hsa-miR-181a, hsa-miR-28, hsa-miR-25, hsa-miR-497, hsa-miR-140, hsa-miR-29c, hsa-miR-181b, hsa-miR-455, hsa-miR-30a; and
a suite of benign miRNA biomarkers selected from hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR- 423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b.

In one embodiment, the kit does not include primers for the amplification of other miRNAs, i.e., miRNAs not enumerated in Table 1, Table 2, Table 3, or Table 4.

In one embodiment, the kit comprises components for the selective quantification of miRNA expression by RNA-Seq methods of miRNAs from
a suite of benign miRNA biomarkers selected from the group consisting of hsa.miR- 211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574; and
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, and hsa.miR.25.

In one embodiment, the kit does not contain components for the selective quantification of expression of other miRNAs by RNA-Seq, i.e. miRNAs not enumerated in Table 1 or Table 2.

In one embodiment, the kit comprises components for the selective quantification of miRNA expression by RNA-Seq of miRNA-miRNa-2, miRNA-31, miRNA-211, miRNA-100, miRNA-125a, and miRNA-125b. In one embodiment, the kit comprises components for the selective quantification of miRNA expression by RNA-Seq of only miRNA-miRNa-2, miRNA-31, miRNA-211, miRNA-100, miRNA-125a, and miRNA-125b.

In one embodiment, the kit comprises components for the selective quantification of miRNA expression by RNA-Seq of miRNAs from
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, hsa.miR.25, hsa-let-7g, hsa-miR-31, hsa-miR-181a, hsa-miR-28, hsa-miR-25, hsa-miR-497, hsa-miR-140, hsa-miR-29c, hsa-miR-181b, hsa-miR-455, hsa-miR-30a; and
a suite of benign miRNA biomarkers selected from the group consisting of hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b.

In one embodiment, the kit does not include components for the selective quantification of expression by RNA-Seq of other miRNAs, i.e., miRNAs not enumerated in Table 1, Table 2, Table 3, or Table 4.

In one embodiment, the kit of the invention comprises one or more tape strips for obtaining a tape strip biopsy of a cutaneous lesion, in combination with components for the measurement of miRNA biomarker expression of a selected suite of miRNAs selected from Table 1 and Table 2, or Table 1 and/or Table 3 and Table 2 and/or Table 4.

### Software and Devices

The generation of a classifier model and application of such model may be performed using a general purpose computer. The classification may be accomplished by deploying computing infrastructure, integrating computer-readable code into a computing system, wherein the computer readable code in combination with the computing system performs the classification. In one embodiment, the scope of the invention encompasses a computer-readable medium tangibly embodying a program of computer-readable instructions executable by a digital processing apparatus to perform the classification methods of the invention, wherein the inputs of the classification are expression values for a selected suite of melanoma miRNA biomarkers and a selected suite of benign miRNA biomarkers.

In one embodiment, the invention comprises a computer readable medium tangibly storing computer-readable instructions which carry out the classification analysis of the invention and provide an output of the classification test. In one embodiment, the invention comprises a kit comprising the aforementioned computer-readable medium and a tape strip for sampling.

### EXAMPLES

### Example 1. A Machine-Learning Classifier Trained with MicroRNA Ratios to Distinguish Melanomas from Nevi

In this study, it was sought to determine whether a miRNA signature can reliably distinguish malignant from benign melanocytic lesions across both published and independently generated datasets.

### Methods

### Meta-Analysis

For meta-analyses all datasets in public databases that contained miRNA profiling for both primary melanoma and nevus samples for comparison were utilized (GSE19229, GSE36236, GSE24996, GSE62372, GSE35579, GSE34460, and E-MTAB-4915). The top differentially expressed miRNAs for each dataset were determined using an FDR cutoff of 0.05 using either microarray and qPCR array data or DeSeq2. To determine overlap, only those miRNAs for which probes were included in every detection platform were considered. Overlap was plotted using the UpsetR package in R.

### Clinical Specimens and Histopathologic Assessment

A training cohort of melanomas with an intact adjacent benign nevus constituted the discovery cohort for this study. Fifteen different areas (8 malignant and 7 benign) from seven cases were selected based upon which samples from a larger published cohort that was previously genetically assessed had leftover material. All cases had previously been retrieved from archive as formalin-fixed paraffin-embedded (FFPE) tissue blocks. Histopathologically distinct areas had been independently evaluated by a panel of 5-8 dermatopathologists for staging. Distinct tumor areas were manually micro-dissected with a scalpel under a dissection scope from unstained tissue sections following the guidance of a pathologist in order to limit stromal cell contamination. Previously, genetic DNA had been isolated from four 10 μM sections. For this study, four additional 20 μM sections were dissected and total RNA was isolated using the RECOVERALL™ Total Nucleic Acid Isolation Kit for FFPE (Ambion Inc.). An independent validation cohort was generated by retrieving obtaining 82 diagnosed melanomas (41 cases) or nevi (41 cases) from UCSF Dermatopathology. Cases were reevaluated by a separate dermatopathologist to confirm diagnosis and obtain histopathological features, but were not excluded for any reason. For RNA isolation of the test cohort, one 20 μM section was scraped off the slide and processed in its entirety without micro-dissection and total RNA was isolated.

MicroRNA-seq and analysis. MicroRNA sequencing libraries were constructed with the using total RNA extracted from FFPE samples. Sequencing was performed on the ILLUMINA HISEQ 2500™ platform at single-end 50 bp. After adaptor sequences were removed, reads were aligned to a human reference (hg37) with Bowtie software and then small RNA reference groups (miRBase21) were counted. Data were submitted to dbGaP (phs001550.v2.p1). Differential expression analysis was performed from feature counts using DeSeq2 software with p-values adjusted for multiple testing with the Benjamin-Hochberg method (p-adj).

### Co-Expression Analysis

Co-expression analysis was restricted to 805 miRNAs with at least one read in at least two samples. Three co-expression networks were identified in R and a minimum 10-member seed and 0.85 correlation threshold. First, pairwise biweight midcorrelation coefficients (cor) were calculated for all possible pairs of miRNAs for all samples. Second, miRNAs were clustered using the hierarchical clustering procedure with complete linkage and 1−cor as a distance measure. The resulting dendrogram was cut at a static height of ˜0.48, corresponding to the top 10% of pairwise correlations for the entire dataset. Third, all clusters consisting of at least 10 members were identified and summarized by their eigengene (i.e. the first principal component obtained via singular value decomposition of the standardized miRNA expression matrix corresponding to each initial cluster) (Horvath and Dong 2008). Fourth, highly similar networks were merged if the Pearson correlation coefficients of their eigengenes exceeded 0.85. This procedure was performed iteratively such that the pair of networks with the highest correlation >0.85 was merged, followed by recalculation of all eigengenes, followed by recalculation of all correlations, until no pairs of networks exceeded the threshold. Following these steps, three co-expression networks were identified by calculating the Pearson correlation between its expression pattern over all samples with each eigengene.

### Linear Discriminant Analysis

To assess and visualize the degree to which the three different miRNA groups can be distinguished based on their expression in the samples, a classifier was constructed that predicted the miRNA network. Linear Discriminant Analysis was used to project the expression of miRNAs in each sample into a two-dimensional linear subspace that optimally separates the different miRNA categories. Subsequently, a support vector machine was trained with the linear kernel to distinguish between the miRNA categories. An area under the ROC curve for categories in a testing set was 0.97, 0.94, and 0.95, respectively.

Gene set enrichment analysis. GSEA was conducted using the three co-expressed miRNA networks as gene sets against public miRNA expression datasets (GSE16368) comparing either primary human melanocytes (GSM817251 GSM1127159, GSM1127164) to keratinocytes (GSM817253, GSM1127111, GSM1127113) or primary human melanocytes to fibroblasts (GSM817252, GSM1127116), for example, as found in NCBI publicly available databases. Positive enrichment of each case corresponded to melanocyte-enriched and negative corresponded to either fibroblast- or keratinocyte-enriched.

### Genomic Analysis

The targeted exon sequencing datasets for each sample of the training cohort were accessed from dbGaP (phs001550.v1.p1). Read alignment, mutational analysis, and copy number analysis was performed. Briefly, sequences were aligned using Burrows-Wheeler Aligner (BWA) with mutational analysis and processing performed using Picard and Genome Analysis Toolkit. Copy number information was obtained with the use of CNVkit. Tumor cell content (tumor cellularity) was calculated bioinformatically using multiple methods when possible, including median mutant allele frequency (MAF) of somatic mutations, MAF of the driver mutation, allelic imbalance over germline and others.

### Classifier Analyses

A feature subset was selected using the Boruta R package (Kursa and Rudnicki 2010. Feature Selection with the Boruta Package. J Stat Softw 36: 1-13) to determine a minimal set of miRNAs for classifier predictive accuracy from the FFPE miRNA-seq data set. Briefly, for each feature (miRNA) present in the RNA-seq data set, a control “shadow-feature” (shadow miR) of comparable expression and variance was generated through random re-assignment of the read counts to different samples. The combined feature set (miRNAs and shadow miRs) was used to train a random forest classifier and the importance of each feature for the accuracy of the model was determined. This process was repeated in 1000 iterations, with miRNAs excluded from each ensuing round once they were significantly less important than the maximum important shadow miR. Thus, the remaining list of miRNAs at the conclusion of the analyses represents only those miRNAs that out-performed an equal number of randomized controls by a statistically significant margin. This initial miRNA list was then further refined by removing miRNAs that were below a minimum expression threshold and/or were not detected across all outside test sets to obtain a final list of 6 miRNAs (miR-211-5p, miR-125a-5p, 125b- 5p, miR-100-5p, miR-31-5p and miR-21-5p). Using log fold-change information from differential expression analysis, each miRNA was associated as melanoma-enriched (ME) or melanoma-depleted/nevus-associated (MD) and miRNA ratios were created from each combination of the 2 ME miRNA (miR-31-5p and miR-21-5p) and 4 MD miRNA (miR-211-5p, miR-125a-5p, 125b-5p, miR-100-5p). A random forest classifier was then built using this transformed minimal signature set and tested by 5-fold repeated cross-validation over 100 repeats to create a final miRNA ratio trained model (MiRTM). The MiRTM was used to classify the datasets described in the meta-analysis where sufficient data were available to obtain sensitivities, specificities, and overall performance by AUC through a ROC curve for each set or combined as a group. The Dadras and Hernando datasets were omitted from the analysis due to insufficient sample sizes (Dadras contained 2 nevus samples), or too many missing features and large sample imbalance (Hernando contained a nevus/mel ratio of 0.1 and many features removed in processing). Similarly, MiRTM was used to classify each case in the validation set with sensitivities and specificities determined using either an optimal threshold based on the Youden index or the sequencing determined threshold 0.5. Overall performance was visualized by the area under a ROC or precision recall curve respectively.

### miRNA qPCR Assay

Total RNA was converted into cDNA. Quantitative PCR for specific miRNA detection was conducted with TAQMAN™ Advanced miRNA Assays (Thermo Fisher).

### Statistical Analysis

Statistical significance was set to 0.05 with p-values adjusted for multiple testing with the Benjamin-Hochberg method. Pearson correlation coefficients were obtained between all continuous features with the equivalent point biserial correlation coefficient for binary variables. Correlation matrices were plotted with the corrplot R Package and correlation plots with the ggpubr R package with 95% confidence intervals calculated for the curves. Sensitivities and specificities were calculated from classification models built using the caret R package. ROC curves were generated using the pROC R package. Confidence intervals (CI) were calculated from the 95% CI of 2000 bootstrap replicates for sensitivity and specificity or the ‘Delong’ method for AUCs using pROC R package. The precision-recall plot was generated using the precrec R package. All data was processed in R (3.3.2)

### RESULTS

### Variation in Tumor Cell Content of FFPE Samples Confounds miRNA Expression Analyses

To identify covariates that could confound differential miRNA expression analyses, a cohort of primary melanomas with intact adjacent benign nevi, from which they arose was utilized. The different progression stages for each sample were diagnostically classified by a panel of at least five dermatopathologists and micro-dissected and genotyped for over five hundred cancer-related genes. Phylogenic trees of the somatic mutations identified in the respective tumor areas were constructed and confirmed the common clonal origin for the different progression stages of each patient. The tumor cell content (referred to herein as tumor cellularity) was calculated using allele frequencies and magnitudes of copy number changes as previously described (Shain et al. 2018 Genomic and Transcriptomic Analysis Reveals Incremental Disruption of Key Signaling Pathways during Melanoma Evolution. Cancer Cell 34: 45-55). Consequently, the dataset was annotated with both clinical features (e.g. patient age, sex, anatomical location of the lesion) as well as genomic information (e.g. mutation burden, copy number variation, tumor cellularity) for each matched pair of nevus and melanoma regions.

To investigate the influence of each genomic and clinical feature on the miRNA expression pattern, miRNA sequencing was conducted on fifteen of the regions from seven cases. In order to first identify potential systemic confounding features, co-expression analyses were first applied for identification of networks of miRNAs sharing similar expression patterns across all regions and identified three co-expression networks that were effectively separated via Linear Discriminate Analysis (LDA) (Langfelder and Horvath 2008 WGCNA: an R package for weighted correlation network analysis. BMC Bioinformatics 9: 559). Each network consists of miRNAs with read counts that are positively correlated across all samples, regardless of level of expression. Next, it was sought to determine whether the expression patterns of these networks correlated with any of the clinical or genomic annotations of the samples, including not only diagnosis but also potentially confounding features, such as patient age. The miRNA expression matrix was summarized for each network by its first principal component and compared these to the sample covariates. Two of the networks (Network 1 and Network 3) were significantly correlated with a diagnosis of melanoma and were not influenced by tumor cellularity or any other clinical feature. Network 1 was also correlated with mutation burden and copy number variation, both measurements of genome damage that increase during progression from nevus to melanoma.

In contrast to the two melanoma-associated networks, Network 2 was positively correlated with tumor cellularity and, to a lesser extent, patient age. This observation suggests that although miRNAs within Network 2 were differentially expressed in melanoma and nevus samples, variation in their observed abundance may reflect the extent of contamination with non-tumor cells rather than different progression stages. Consistent with this interpretation, it was observed that miRNAs known to be expressed in cultured primary human keratinocytes were enriched in Network 2 as would be expected if keratinocytes were a significant fraction of contaminating non-tumor cells. Conversely, miRNAs known to be expressed in cultured primary human melanocytes were enriched in Network 3 consistent with changes in Network 3 reflecting melanocyte biology. Together, these data suggest that miRNA profiling datasets derived from micro-dissected FFPE samples can contain sufficient levels of contaminating non-tumor cells to influence the overall miRNA expression profile. Contamination by non-tumor cells is expected to vary among samples dependent on their size, histologic type (predominantly junctional versus intradermal), and preparation (e.g. precision of microdissection). If not controlled for, variation in tumor cellularity was expected to degrade the reproducibility of signatures across studies. Indeed, miRNAs from Network 2 constituted up to thirty percent of the miRNAs in expression signatures reported from the seven previously reported datasets. This result highlights the need for alternative analytical methods for identifying miRNA signatures predictive of melanoma diagnosis from FFPE derived samples.

### Classification of Nevus from Melanoma Samples with miRNA Ratios

To identify miRNAs that best distinguish malignant from benign melanocytic lesions, feature selection (FS) was utilized. FS is a method of machine learning that is frequently used to simplify predictive models and to avoid analytical pitfalls such as the phenomena of over-fitting and the ‘curse of dimensionality’. In the context of biology, FS methods can be applied to gene expression datasets to identify sets of features (in this case, miRNAs) that are more often biologically relevant and ultimately improve classification performance. There are common FS methods, such as univariate statistical test filtering (e.g. FDR, t-test) and feature rank wrappers (e.g. backward selection) that will identify individual features that are independently relevant, but they may miss features that are only relevant in the context of complex networks. An alternative strategy is the all-relevant features (ARF) selection approach that involves multiple iterations of feature ranking and can determine both weak and strong relevant features (Kursa and Rudnicki 2010). An ARF method called Boruta was used to obtain an initial list of those miRNAs that were most important for discriminating the benign and malignant regions from the cohort across 1000 random forest iterations. All miRNAs with more than five total reads were considered, resulting in 341 unique features. For each miRNA, a second artificial feature was generated through randomized redistribution of the read counts across samples. These ‘shadow features’ provided an equal number of negative control features for which to compare each experimental feature. Boruta was conducted with the combined 682 experimental and negative control features, ranking the importance of each feature for the accurate classification of nevus samples from melanoma samples with each iteration. 38 miRNAs were identified that ranked higher than the maximum-performing shadow feature with a p-value of less than 0.001. To enable comparison with published studies, it was also required that the expression levels of each miRNA were assessed in all published datasets. The final list of feature-selected miRNAs contained two miRNAs with increased expression (miR-31-5p, miR-21-5p) and four miRNAs (miR-211-5p, miR-125a-5p, miR-125b-5p, miR-100-5p) with decreased expression in melanomas. These miRNAs are referred to as melanoma-enriched and melanoma-depleted miRNAs, respectively.

The use of transcript ratios has been previously demonstrated to strengthen the prediction accuracy through simplification of features. A diagnostic score was developed using all ratios of melanoma-enriched miRNAs to melanoma-depleted miRNAs. This approach controls for variations in lesion composition (e.g. the relative amount of malignant and benign tissue) when micro-dissection boundaries are not known and differences in tumor cellularity when genetic data are not available. This approach also normalizes for the fraction of miRNAs of melanocytic origin (as opposed to the totality of miRNAs), amplifies the signal from malignant cells by normalizing melanoma-enriched miRNAs to melanoma-depleted miRNAs, and permits cross-platform comparisons without the need for cross-platform normalization. Expression levels for each of the two melanoma-enriched miRNAs was divided by each of the four melanoma-depleted miRNAs, producing eight miRNA ratios (miR-31-5p/miR-211-5p, miR-31-5p/miR-125a-5p, miR-31-5p/miR-125b-5p, miR-31-5p/miR-100-5p, miR-21-5p/miR-211-5p, miR-21-5p/miR-125a-5p, miR-21-5p/miR-125b-5p, and miR-21-5p/miR-100-5p). These eight ratios were used to train a random forest classifier. The final miRNA Ratio Trained Model (MiRTM) resulted in an area under receiver operating characteristic curves (AUC) of 1.0 for the discovery set of 7 samples containing 15 matched melanoma and nevus regions.

Validation of the MiRTM on previously published datasets. To test the accuracy of the MiRTM on independent datasets we obtained and combined the raw data from five previously published miRNA profiling studies (Sand, Xu, Chen, Komina & Jukic, as cited herein). These studies contained both microarray and RT-qPCR datasets. Regardless of the platform, the eight miRNA expression ratios were used as input for the model trained on the sequencing data. The MiRTM resulted in an AUC of ROC of 0.92 and an AUC of Precision-Recall of 0.95. Two thresholds were set to calculate sensitivity and specificity. First, the optimal threshold was used as defined by the sequencing cohort (0.5), which resulted in a sensitivity of 0.83 and a specificity of 0.83. As this validation cohort was constructed using different technical platforms for miRNA profiling, sensitivity and specificity were also calculated using the optimal threshold for the ROC curve as 0.94 and 0.83 respectively.

To further examine the reproducibility of the model, MiRTM was run for each individual previously published dataset. Across all datasets, the MiRTM resulted in an AUC of 0.8 or higher, with an average of 0.922. Optimal sensitivity remained above 0.9 across each dataset. With the exception of one dataset, optimal specificity was above 0.8. Interestingly, that dataset contained the highest level of the miRNA network identified as associated with tumor cellularity.

### Validation of the MiRTM on Randomly Selected Cases

Discovery phase cohorts are often selected for unambiguous and homogenous cases. To further validate the model on a greater diversity of cases, 82 biopsied melanocytic lesions were randomly retrieved—41 neoplasms diagnosed as nevi and 41 diagnosed as melanoma—from the archives of the UCSF Dermatopathology Section. All diagnoses were reviewed and confirmed by an independent dermatopathologist. This cohort contained a greater range of tumor cellularity and subtypes of melanocytic neoplasms than the discovery cohort. Instead of micro-dissection, entire FFPE sections were scraped to obtain bulk RNA. The abundance of the six miRNAs was assessed by RT-qPCR. C values were converted to expression ratios using the linear

### Validation of the MiRTM on Randomly Selected Cases

Discovery phase cohorts are often selected for unambiguous and homogenous cases. To further validate the model on a greater diversity of cases, 82 biopsied melanocytic lesions were randomly retrieved—41 neoplasms diagnosed as nevi and 41 diagnosed as melanoma—from the archives of the UCSF Dermatopathology Section. All diagnoses were reviewed and confirmed by an independent dermatopathologist. This cohort contained a greater range of tumor cellularity and subtypes of melanocytic neoplasms than the discovery cohort. Instead of micro-dissection, entire FFPE sections were scraped to obtain bulk RNA. The abundance of the six miRNAs was assessed by RT-qPCR. C values were converted to expression ratios using the linear transformation (2\hat{ }−C). The resultant AUC of ROC for the unfiltered cohort (UC) was 0.92. The AUC of the Precision-Recall curve was 0.911. To classify the lesions into benign and malignant, we again considered two thresholds. Using the threshold defined by our sequencing cohort (0.5) the model achieved sensitivity of 0.83 and specificity of 0.71. The optimal sensitivity and specificity of this ROC curve were 0.81 and 0.90.

As the MiRTM demonstrated a lower sensitivity for the more diverse second validation cohort compared to the previously published cohorts, it was examined whether the model was affected by tumor cellularity and patient age. When the MiRTM score was compared to the tumor cellularity in each sample as assessed by a dermatopathologist, no correlation was found, suggesting that the MiRTM score was unaffected by this variable. Similarly, no correlation was found between MiRTM score and age . To investigate whether other features might influence the MiRTM score, the correlations between fourteen clinical features and the MiRTM score were calculated. In the melanoma samples, it was observed the expected correlations between clinical features such as age and solar elastosis as a proxy for mutation burden. However, no significant correlations were observed with the MiRTM score that suggested any of the features other than diagnosis could influence the score. For example, changes in the thickness or size of the lesion did not affect the MiRTM score. Similarly, in the nevus samples, most features did not influence the score, including the presence of dysplastic features.

### Discussion

Numerous studies have analyzed miRNA expression at different stages of melanoma progression, collectively identifying over 500 miRNAs enriched in nevi or melanomas. The analysis described herein has refined this expansive list to six miRNAs that reproducibly distinguish nevi from melanoma across independent datasets and profiling platforms. This signature was identified by controlling two important variables, interobserver variability of diagnosis and variability in tumor cellularity. To address the first variable, a discovery cohort was used for which the diagnosis accuracy was established by requiring the median concordance among five to eight different dermatopathologists and supporting genetic features, eliminating the possibility of training the model with misdiagnosed cases. Secondly, melanomas and nevi were matched as pairs representing different progression stages of the same neoplastic clone, eliminating variability by comparing different lesions from different patients. Thirdly, miRNAs from the signature, whose expression is influenced by tumor cellularity were identified and excluded, thereby removing a covariate that has confounded previous analyses. When tested on six datasets assembled by independent groups, a model trained on expression ratios of the refined signature classified benign from malignant melanocytic lesions with an average AUC of ROC above 0.91.

The performance of the MiRTM is comparable to other molecular tests for distinguishing benign melanocytic nevi from melanoma, including chromosomal analysis by fluorescence in situ hybridization (sensitivity 0.72-1.00, specificity 0.90-1.00) and melanoma gene expression profiling (sensitivity 0.63-0.90, specificity 0.88-0.93). The MiRTM does not perform as well as chromosomal analysis by array comparative genomic hybridization (aCGH, sensitivity 0.92-0.96, specificity 0.87-1.00). However, assessment by the MiRTM requires only a single section of FFPE material, does not require microdissection and RT-qPCR is a quick and affordable assay making this approach a candidate for lesions where tissue availability is limited.

The only measured feature that correlated with the MiRTM score overall was diagnosis. However, in the benign samples of the validation set, it was observed that inflammation can result in false-positive calls. Indeed, some of the miRNAs (miR-125b, miR-31, miR-21) have been associated with inflammation in psoriasis. However, inflammation was not correlated with the MiRTM score over all samples in the cohort suggesting the feature-selected miRNAs are not exclusively an inflammation signature.

Of the six miRNAs of the signature, three (miR-211-5p, miR-21-5p, and miR-125b-5p) have been linked to melanoma, have been previously validated by in situ hybridization, and have been functionally assessed in melanoma cell lines. MiR-21 is an established oncomir and regulates genes involved in increased proliferation and invasion. It is upregulated in many cancers including melanoma and its expression correlates with progression from nevi to primary melanomas and then to metastatic melanomas. Conversely, miR-125b is often downregulated in cancers, including advanced melanomas, where its loss results in increased expression of cJUN and MLK3. MiR-211 is among the most well-established functional miRNAs in melanocytes and is downstream of the important melanocyte lineage transcription factor MITF. It is often downregulated during melanoma progression and has been linked to invasion through regulation of BRN2, NFAT5 and TGFβPR2. The other miRNAs in the signature are less well characterized in melanocytes. As another miR-125 family member, miR-125a is expected to target a similar set of genes as miR-125b, but has been mostly described in other cancers. MiR-31 is upregulated in some cancers, but its role as an obligate oncomir is controversial as it is transcribed from a commonly deleted or methylated genomic region in many cancers. Similarly, miR-100 has also been described as both a tumor suppressor and an oncomir depending on the context (Li et al. 2015). Regardless of their precise functional role in the context of melanocytic neoplasia, the analyses demonstrate that the relative expression ratios of these six miRNAs can assist in distinguishing benign melanocytic nevi from malignant melanoma in FFPE samples.

1-25. (canceled)

26. A method of treating melanoma in a subject, the method of treatment comprising:
a sample is collected from the subject comprising cutaneous lesion tissue;
the sample is assayed to assess the expression value of a selected panel of benign lesion miRNA biomarkers
the sample is assayed to assess the expression value of a panel of selected melanoma miRNA biomarkers;
the measured expression values of the selected melanoma miRNA biomarkers and the selected benign lesion miRNA biomarkers are input to a classifier model;
a classification of the sample as melanoma or benign is output by the model; and
an appropriate treatment is administered to the subject if the sample is classified as melanoma.

27. The method of claim 26, wherein
the panel of benign lesion miRNA biomarkers comprises one or more biomarkers selected from the group consisting of hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574; and
the panel of melanoma miRNA biomarkers comprises one or more biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, and hsa.miR.25.

28. The method of claim 27, wherein
the panel of selected benign lesion miRNA biomarkers comprises miRNA-211, miRNA-100, miRNA-125a, and miRNA-125b; and
the panel of selected melanoma miRNA biomarkers comprises miRNa-21 and miRNA-31.

29. The method of claim 26, wherein
the panel of selected benign lesion miRNA biomarkers consists of miRNA-211, miRNA-100, miRNA-125a, and miRNA-125b; and
the panel of selected melanoma miRNA biomarkers consists of miRNa-21 and miRNA-31.

30. The method of claim 26, wherein
the classifier is a transcript ratio classifier, wherein the inputs are the ratio of miR-31 expression to miR-211 expression; the ratio of miR-31 expression to miR-125a expression; the ratio of miR-31 expression to miR-125b expression; the ratio of, miR-31 expression to miR-100 expression; the ratio of miR-21 expression to miR-211 expression; the ratio of miR-21 expression to miR-125a expression; the ratio of miR-21 expression to miR-125b expression; and the ratio of miR-21 expression to miR-100 expression.

31. The method of claim 26, wherein
the sample comprises a tape-strip biopsy sample.

32. The method of claim 31, wherein the
the panel of selected benign lesion miRNA biomarkers comprises one or more biomarkers selected from the group consisting of hsa.miR-211, hsa.miR-328, hsa.miR-125a, hsa.miR-125b, hsa.miR-let7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b; and
the panel of selected melanoma miRNA biomarkers comprises one or more biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR-106b, hsa.miR-148b, hsa.miR-103a, hsa.miR.20a, hsa.miR.25, hsa-let-7g, hsa-miR-31, hsa-miR-181a, hsa-miR-28, hsa-miR-25, hsa-miR-497, hsa-miR-140, hsa-miR-29c, hsa-miR-181b, hsa-miR-455, and hsa-miR-30a.

33. The method of claim 26, wherein
the selected treatment is a treatment selected from the group consisting of staging of the melanoma, assessment of metastasis, surgical removal of the lesion and surrounding tissue, adjuvant treatment, chemotherapy, radiation, and/or immunotherapy.

34. A kit comprising
components for the measurement of miRNA biomarker expression of
a suite of benign miRNA biomarkers selected from the group consisting of hsa.miR-211, hsa.miR-328, hsa.miR-125a, hsa.miR-125b, hsa.miR-1et7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574; and
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR- hsa.miR-142, hsa.miR-9, hsa.miR-509, hsa.miR-106b, hsa.miR-148b, hsa.miR-155, 103a, hsa.miR.20a, and hsa.miR.25.

35. The kit of claim 34, wherein
the kit comprises components for the measurement of miRNa-21 and miRNA-31, miRNA-211, miRNA-100, miRNA-125a, and miRNA-125b.

36. The kit of claim 34, wherein
the kit comprises components for the measurement of only miRNa-21 and miRNA-31, miRNA-211, miRNA-100, miRNA-125a, and miRNA-125b.

37. The kit of claim 34, wherein
the components comprises a microarray comprising probes that selectively hybridize with the selected miRNAs.

38. The kit of claim 34, wherein
the components comprises PCR primers for the selective amplification of the selected miRNAs.

39. A kit, comprising
one or more tape strips for obtaining a tape strip biopsy of a cutaneous lesion; and
components for the measurement of miRNA biomarker expression of
a suite of melanoma miRNA biomarkers selected from the group consisting of hsa.miR-17, hsa.miR-93, hsa.miR-107, hsa.miR-22, hsa.miR-31, hsa.miR-98, hsa.miR-21, hsa.miR-128, hsa.miR-185, hsa.miR-7, hsa.miR-15a, hsa.miR-16, hsa.miR-27a, hsa.miR-155, hsa.miR-142, hsa.miR-9, hsa.miR-509, hsa.miR- hsa.miR-148b, hsa.miR-103a, hsa.miR.20a, hsa.miR.25, hsa-let-7g, hsa-106b, miR-31, hsa-miR-181a, hsa-miR-28, hsa-miR-25, hsa-miR-497, hsa-miR-140, hsa-miR-29c, hsa-miR-181b, hsa-miR-455, hsa-miR-30a; and
a suite of benign miRNA biomarkers selected from hsa.miR-211, hsa.miR-328, hsa.miR-125a, hsa.miR-125b, hsa.miR-1et7c., hsa.miR-100, hsa.miR-let.7b, hsa.miR-214, hsa.miR-193a, hsa.miR-6087, hsa.miR-320a, hsa.miR-423, hsa.miR-let.7d, hsa.miR-let.7e, hsa.miR-197, hsa.miR-99b, hsa.miR-532, and hsa.miR-574, hsa-miR-3141, hsa-miR-6087, hsa-miR-5010, hsa-miR-7847, hsa-miR-6089, hsa-miR-5100, hsa-miR-1246, hsa-miR-3960, hsa-miR-6515, hsa-miR-6734, hsa-miR-4466, hsa-miR-193b, hsa-miR-4516, hsa-miR-5187, hsa-miR-150, hsa-miR-7704, hsa-miR-4492, hsa-miR-6512, hsa-miR-4532, hsa-miR-4497, hsa-miR-296, hsa-miR-1290, hsa-miR-30c-1, hsa-miR-342, hsa-miR-3196, hsa-miR-629, hsa-miR-4429, hsa-miR-130b, and hsa-let-7b.

40. The kit of claim 39, wherein
the components comprises a microarray comprising probes that selectively hybridize with the selected miRNAs.

41. The kit of claim 40, wherein
the components comprises PCR primers for the selective amplification of the selected miRNAs.
